(19) United States
(12) Patent Application Publication
(10) Pub. No.: US 2020/0237562 A1
(43) Pub. Date: Jul. 30, 2020

(54) VARIABLE DENSITY DRESSING

(71) Applicant: KCI Licensing, Inc., San Antonio, TX (US)

(72) Inventors: Justin RICE, San Antonio, TX (US); Brett L. MOORE, San Antonio, TX (US); Christopher Allen CARROLL, San Antonio, TX (US); Justin Alexander LONG, Lago Vista, TX (US)

(21) Appl. No.: 16/745,075

(22) Filed: Jan. 16, 2020

Related U.S. Application Data
(60) Provisional application No. 62/796,407, filed on Jan. 24, 2019.

(51) Int. Cl. A61F 13/00; A61F 13/02

(52) U.S. Cl. CPC A61F 13/00068; A61L 2300/402; A61F 2013/00927; A61F 13/0276

(57) ABSTRACT

A dressing for treating a tissue site with negative pressure, methods of manufacturing the dressing, and methods of using the dressing are described. The dressing includes a debriding manifold having a plurality of first regions with a first density, and a plurality of second regions with a second density less than the first density. The dressing can include a cover configured to be disposed over the debriding manifold. The cover includes a perimeter extending beyond the debriding manifold.

### RELATED APPLICATION

The present invention claims the benefit, under 35 USC § 119(e), of the filing of U.S. Provisional Patent Application No. 62/796,407, filed Jan. 24, 2019, which is incorporated herein by reference for all purposes.

### TECHNICAL FIELD

The invention set forth in the appended claims relates generally to tissue treatment systems and more particularly, but without limitation, to dressings for treatment with negative-pressure.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for debriding tissue in a negative-pressure therapy environment are set forth in the appended claims. Illustrative embodiments are also provided to enable a person skilled in the art to make and use the claimed subject matter.

For example, in some embodiments, a dressing for treating a tissue site with negative pressure is described. The dressing can include a debriding manifold comprising a plurality of first regions having a first density, and a plurality of second regions having a second density less than the first density. The dressing can also include a cover configured to be disposed over the debriding manifold and comprising a perimeter extending beyond the debriding manifold.

In some embodiments, the second regions are recessed relative to the first regions. In other embodiments, the first regions comprise a first material and the second regions comprise a second material. The plurality of first regions and the plurality of second regions can be alternately distributed in an array through the debriding manifold. The debriding manifold can comprise foam, open-cell foam, or reticulated foam.

In some embodiments, the dressing can include a support layer configured to be disposed between the debriding manifold and the cover. The dressing can also include a buffer layer having a first side and a second side, the first side disposed adjacent to the debriding manifold and the second side configured to face the tissue site. The buffer layer can be laminated to the second side of the debriding manifold. The buffer layer can be configured to resist ingrowth from the tissue site. The buffer layer can be perforated and be formed from polyurethane or polyethylene, and at least partially infused with citric acid, silver nitrate, or a pain-relieving agent that can be lidocaine or ketoprophen.

More generally, a method of manufacturing a dressing for negative-pressure treatment is described. A manifold having a first side and a second side can be provided. A first wave pattern can be cut into the second side of the manifold. The manifold can be rotated ninety degrees, and a second wave pattern can be cut into the second side of the manifold. The manifold can be simultaneously compressed and heated on at least the second side of the manifold.

In some embodiments, the manifold comprises foam. In some embodiments, the first wave pattern and the second wave pattern are a square wave pattern. In other embodiments, the first wave pattern and the second wave pattern are a triangular wave pattern. In still other embodiments, the first wave pattern and the second wave pattern are a sine wave pattern.

Alternatively, other example embodiments may describe a dressing for treating a tissue site with negative pressure. The dressing can include a debridement manifold having a first section and a second section, the second section positioned adjacent to the first section. The second section of the debridement manifold can comprise a plurality of first regions and a plurality of second regions, the plurality of first regions having a greater density than the plurality of second regions.

In some embodiments, the first section of the debridement manifold is a first layer and the second section of the debridement manifold is a second layer. The first layer can have a first side and a second side, and the second side can be configured to face the second layer. The second side of the first layer comprises a first plurality of protrusions. The second layer can have a first side and a second side, and the first side can be configured to face the second side of the first layer. In some embodiments, the plurality of first regions and the plurality of second regions are square-shaped. In other embodiments, the plurality of first regions and the plurality of second regions are triangle-shaped. In still other embodiments, the plurality of first regions and the plurality of second regions are wave-shaped.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure treatment in accordance with this specification;

FIG. 2 is a plan view illustrating additional details of a tissue interface that may be associated with some embodiments of the therapy system of FIG. 1;

FIG. 3 is a plan view illustrating additional details of the tissue interface of FIG. 2 disposed in an exemplary bench testing apparatus in accordance with some embodiments of the therapy system of FIG. 1;

FIG. 4 is a bottom perspective view illustrating additional details that may be associated with bench testing of the tissue interface of FIG. 3 under negative pressure;

FIG. 5 is a side view illustrating additional details that may be associated with bench testing of the tissue interface of FIG. 3 under negative pressure;

FIG. 6 is a perspective view illustrating additional details that may be associated with the tissue interface of FIG. 2 disposed in an exemplary bench testing apparatus in accordance with some embodiments of the therapy system of FIG. 1;

FIG. 7 is a perspective view illustrating additional details that may be associated with the tissue interface of FIG. 2 and a rigid layer disposed in the exemplary bench testing apparatus of FIG. 6;

FIG. 8 is a perspective view illustrating additional details that may be associated with the use of the tissue interface of FIG. 2;

FIG. 9 is a side view illustrating additional details of another tissue interface that may be associated with some embodiments of the therapy system of FIG. 1;

FIG. 10 is a bottom plan view illustrating additional details of the tissue interface of FIG. 9;

FIG. 11 is a sectional view illustrating additional details that may be associated with the tissue interface of FIG. 9 disposed at a tissue site;

FIG. 12 is a sectional view illustrating additional details that may be associated with the tissue interface of FIG. 9 disposed at a tissue site under negative pressure;

FIG. 13 is a bottom plan view illustrating additional details of another tissue interface that may be associated with some embodiments of the therapy system of FIG. 1;

FIG. 14 is a bottom plan view illustrating additional details of another tissue interface that may be associated with some embodiments of the therapy system of FIG. 1;

FIG. 15 is a bottom plan view illustrating additional details associated with a manufacturing process of a tissue interface that may be associated with some embodiments of the therapy system of FIG. 1;

FIG. 16 is a sectional view taken along line 16-16 of FIG. 15 illustrating additional details associated with a manufacturing process for the tissue interface of FIG. 15;

FIG. 17 is a bottom plan view illustrating additional details associated with a manufacturing process for the tissue interface of FIG. 15;

FIG. 18 is a bottom plan view illustrating additional details associated with a manufacturing process for the tissue interface of FIG. 15;

FIG. 19 is a sectional view taken along line 19-19 of FIG. 18 illustrating additional details associated with a manufacturing process for the tissue interface of FIG. 15;

FIG. 20 is a bottom plan view illustrating additional details associated with a manufacturing process for the tissue interface of FIG. 15;

FIG. 21 is a sectional view taken along line 21-21 of FIG. 20 illustrating additional details associated with a manufacturing process for the tissue interface of FIG. 15; and

FIG. 22 is a perspective assembly view illustrating additional details associated with another embodiment of a tissue interface that can be used with some embodiments of the therapy system of FIG. 1.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective but are instead areas in which it may be desirable to add or promote the growth of additional tissue. For example, negative pressure may be applied to a tissue site to grow additional tissue that may be harvested and transplanted.

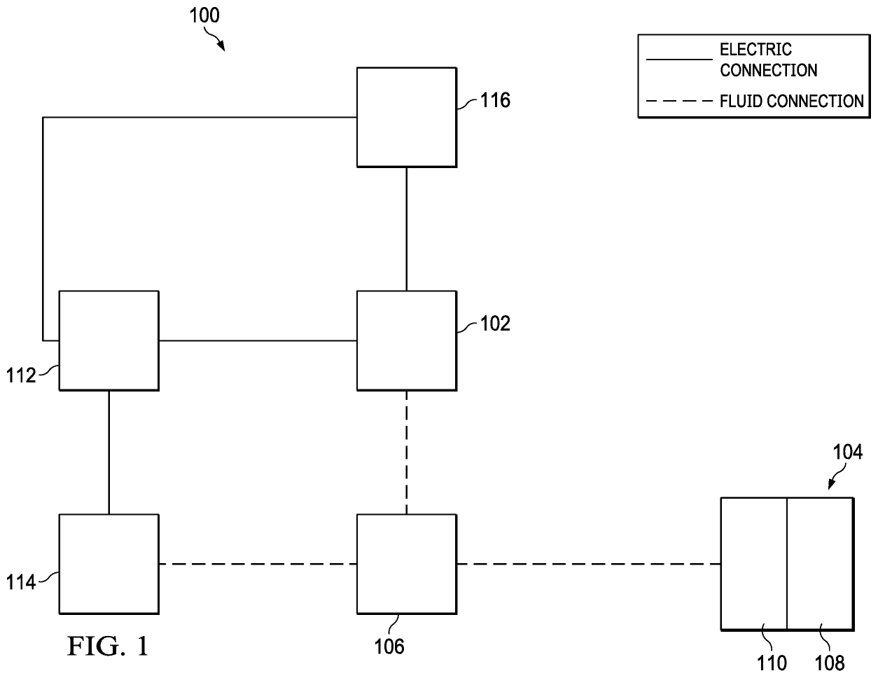

FIG. 1 is a simplified functional block diagram of an example embodiment of a therapy system 100 that can provide negative-pressure therapy to a tissue site in accordance with this specification. The therapy system 100 may include a source or supply of negative pressure, such as a negative-pressure source 102, and one or more distribution components. A distribution component is preferably detachable and may be disposable, reusable, or recyclable. A dressing, such as a dressing 104, and a fluid container, such as a container 106, are examples of distribution components that may be associated with some examples of the therapy system 100. As illustrated in the example of FIG. 1, the dressing 104 may comprise or consist essentially of a tissue interface 108, a cover 110, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 104. For example, such a dressing interface may be a SENSAT.R.A.C.™ Pad available from Kinetic Concepts, Inc. of San Antonio, Tex.

The therapy system 100 may also include a regulator or controller, such as a controller 112. Additionally, the therapy system 100 may include sensors to measure operating parameters and provide feedback signals to the controller 112 indicative of the operating parameters. As illustrated in FIG. 1, for example, the therapy system 100 may include a first sensor 114 and a second sensor 116 coupled to the controller 112.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 102 may be combined with the controller 112 and other components into a therapy unit.

In general, components of the therapy system 100 may be coupled directly or indirectly. For example, the negative-pressure source 102 may be directly coupled to the container 106 and may be indirectly coupled to the dressing 104 through the container 106. Coupling may include fluid, mechanical, thermal, electrical, or chemical coupling (such as a chemical bond), or some combination of coupling in some contexts. For example, the negative-pressure source 102 may be electrically coupled to the controller 112 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 102, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 102 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 106 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 112, may be a microprocessor or computer programmed to operate one or more components of the therapy system 100, such as the negative-pressure source 102. In some embodiments, for example, the controller 112 may be a microcontroller, which generally comprises an integrated circuit containing a processor core and a memory programmed to directly or indirectly control one or more operating parameters of the therapy system 100. Operating parameters may include the power applied to the negative-pressure source 102, the pressure generated by the negative-pressure source 102, or the pressure distributed to the tissue interface 108, for example. The controller 112 is also preferably configured to receive one or more input signals, such as a feedback signal, and programmed to modify one or more operating parameters based on the input signals.

Sensors, such as the first sensor 114 and the second sensor 116, are generally known in the art as any apparatus operable to detect or measure a physical phenomenon or property, and generally provide a signal indicative of the phenomenon or property that is detected or measured. For example, the first sensor 114 and the second sensor 116 may be configured to measure one or more operating parameters of the therapy system 100. In some embodiments, the first sensor 114 may be a transducer configured to measure pressure in a pneumatic pathway and convert the measurement to a signal indicative of the pressure measured. In some embodiments, the first sensor 114 may be a piezo-resistive strain gauge. The second sensor 116 may optionally measure operating parameters of the negative-pressure source 102, such as a voltage or current, in some embodiments. Preferably, the signals from the first sensor 114 and the second sensor 116 are suitable as an input signal to the controller 112, but some signal conditioning may be appropriate in some embodiments. For example, the signal may need to be filtered or amplified before it can be processed by the controller 112. Typically, the signal is an electrical signal, but may be represented in other forms, such as an optical signal or a pneumatic signal.

The tissue interface 108 can be generally adapted to partially or fully contact a tissue site. The tissue interface 108 may take many forms, and may have many sizes, shapes, or thicknesses, depending on a variety of factors, such as the type of treatment being implemented or the nature and size of a tissue site. For example, the size and shape of the tissue interface 108 may be adapted to the contours of deep and irregular shaped tissue sites. Any or all of the surfaces of the tissue interface 108 may have an uneven, a coarse, or a jagged profile.

In some embodiments, the tissue interface 108 may comprise or consist essentially of a manifold. A manifold in this context may comprise or consist essentially of a means for collecting or distributing fluid across the tissue interface 108 under pressure. For example, a manifold may be adapted to receive negative pressure from a source and distribute negative pressure through multiple apertures across the tissue interface 108, which may have the effect of collecting fluid from across a tissue site and drawing the fluid toward the source. In some embodiments, the fluid path may be reversed, or a secondary fluid path may be provided to facilitate delivering fluid across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 108 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 108 may also vary according to needs of a prescribed therapy. The 25% compression load deflection of the tissue interface 108 may be at least 0.35 pounds per square inch, and the 65% compression load deflection may be at least 0.43 pounds per square inch. In some embodiments, the tensile strength of the tissue interface 108 may be at least 10 pounds per square inch. The tissue interface 108 may have a tear strength of at least 2.5 pounds per inch. In some embodiments, the tissue interface 108 may be foam comprised of polyols such as polyester or polyether, isocyanate such as toluene diisocyanate, and polymerization modifiers such as amines and tin compounds. In some examples, the tissue interface 108 may be reticulated polyurethane foam such as found in V.A.C.® GRANUFOAM™ dressing or V.A.C. VERAFLO™ dressing, both available from Kinetic Concepts, Inc. of San Antonio, Tex.

The thickness of the tissue interface 108 may also vary according to needs of a prescribed therapy. For example, the thickness of the tissue interface 108 may be decreased to reduce tension on peripheral tissue. The thickness of the tissue interface 108 can also affect the conformability of the tissue interface 108. In some embodiments, a thickness in a range of about 5 millimeters to 10 millimeters may be suitable.

The tissue interface 108 may be either hydrophobic or hydrophilic. In an example in which the tissue interface 108 may be hydrophilic, the tissue interface 108 may also wick fluid away from a tissue site, while continuing to distribute negative pressure to the tissue site. The wicking properties of the tissue interface 108 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity.

In some embodiments, the tissue interface 108 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 108 may further serve as a scaffold for new cell-growth, or a scaffold material may be used in conjunction with the tissue interface 108 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 110 may provide a bacterial barrier and protection from physical trauma. The cover 110 may also be constructed from a material that can reduce evaporative losses and provide a fluid seal between two components or two environments, such as between a therapeutic environment and a local external environment. The cover 110 may comprise or consist of, for example, an elastomeric film or membrane that can provide a seal adequate to maintain a negative pressure at a tissue site for a given negative-pressure source. The cover 110 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 110 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 110 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minn.; polyurethane (PU) drape, commercially available from Avery Dennison Corporation, Pasadena, Calif.; polyether block polyamide copolymer (PEBAX), for example, from Arkema S.A., Colombes, France; and Inspire 2301 and Inpsire 2327 polyurethane films, commercially available from Coveris Advanced Coatings, Wrexham, United Kingdom. In some embodiments, the cover 110 may comprise INSPIRE 2301 having an MVTR (upright cup technique) of 2600 g/m²/24 hours and a thickness of about 30 microns.

An attachment device may be used to attach the cover 110 to an attachment surface, such as undamaged epidermis, a gasket, or another cover. The attachment device may take many forms. For example, an attachment device may be a medically-acceptable, pressure-sensitive adhesive configured to bond the cover 110 to epidermis around a tissue site. In some embodiments, some or all of the cover 110 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

In operation, the tissue interface 108 may be placed within, over, on, or otherwise proximate to a tissue site. If the tissue site is a wound, for example, the tissue interface 108 may partially or completely fill the wound, or it may be placed over the wound. The cover 110 may be placed over the tissue interface 108 and sealed to an attachment surface near a tissue site. For example, the cover 110 may be sealed to undamaged epidermis peripheral to a tissue site. Thus, the dressing 104 can provide a sealed therapeutic environment proximate to a tissue site, substantially isolated from the external environment, and the negative-pressure source 102 can reduce pressure in the sealed therapeutic environment.

The fluid mechanics of using a negative-pressure source to reduce pressure in another component or location, such as within a sealed therapeutic environment, can be mathematically complex. However, the basic principles of fluid mechanics applicable to negative-pressure therapy are generally well-known to those skilled in the art, and the process of reducing pressure may be described illustratively herein as “delivering,” “distributing,” or “generating” negative pressure, for example.

In general, exudates and other fluids flow toward lower pressure along a fluid path. Thus, the term “downstream” typically implies a position in a fluid path relatively closer to a source of negative pressure or further away from a source of positive pressure. Conversely, the term “upstream” implies a position relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site through the tissue interface 108 in the sealed therapeutic environment can induce macro-strain and micro-strain in the tissue site. Negative pressure can also remove exudate and other fluid from a tissue site, which can be collected in container 106.

In some embodiments, the controller 112 may receive and process data from one or more sensors, such as the first sensor 114. The controller 112 may also control the operation of one or more components of the therapy system 100 to manage the pressure delivered to the tissue interface 108. In some embodiments, the controller 112 may include an input for receiving a desired target pressure and may be programmed for processing data relating to the setting and inputting of the target pressure to be applied to the tissue interface 108. In some example embodiments, the target pressure may be a fixed pressure value set by an operator as the target negative pressure desired for therapy at a tissue site and then provided as input to the controller 112. The target pressure may vary from tissue site to tissue site based on the type of tissue forming a tissue site, the type of injury or wound (if any), the medical condition of the patient, and the preference of the attending physician. After selecting a desired target pressure, the controller 112 can operate the negative-pressure source 102 in one or more control modes based on the target pressure and may receive feedback from one or more sensors to maintain the target pressure at the tissue interface 108.

In some embodiments, the controller 112 may have a continuous pressure mode, in which the negative-pressure source 102 is operated to provide a constant target negative pressure for the duration of treatment or until manually deactivated. Additionally or alternatively, the controller may have an intermittent pressure mode. For example, the controller 112 can operate the negative-pressure source 102 to cycle between a target pressure and atmospheric pressure. For example, the target pressure may be set at a value of 135 mmHg for a specified period of time (e.g., 5 min), followed by a specified period of time (e.g., 2 min) of deactivation. The cycle can be repeated by activating the negative-pressure source 102, which can form a square wave pattern between the target pressure and atmospheric pressure. In some embodiments, the controller 112 may control or determine a variable target pressure in a dynamic pressure mode, and the variable target pressure may vary between a maximum and minimum pressure value that may be set as an input prescribed by an operator as the range of desired negative pressure. The variable target pressure may also be processed and controlled by the controller 112, which can vary the target pressure according to a predetermined waveform, such as a triangular waveform, a sine waveform, or a saw-tooth waveform. In some embodiments, the waveform may be set by an operator as the predetermined or time-varying negative pressure desired for therapy.

Some tissue sites may not heal according to the normal medical protocol and may develop areas of necrotic tissue. Necrotic tissue may be dead tissue resulting from infection, toxins, or trauma that caused the tissue to die faster than the tissue can be removed by the normal body processes that regulate the removal of dead tissue. Sometimes, necrotic tissue may be in the form of slough, which may include a viscous liquid mass of tissue. Generally, slough is produced by bacterial and fungal infections that stimulate an inflammatory response in the tissue. Slough may be a creamy yellow color and may also be referred to as pus. Eschar may be a portion of necrotic tissue that has become dehydrated and hardened. Eschar may be the result of a burn injury, gangrene, ulcers, fungal infections, spider bites, or anthrax. Eschar may be difficult to move without the use of surgical cutting instruments. Necrotic tissue can also include thick exudate and fibrinous slough.

If a tissue site develops necrotic tissue, the tissue site may be treated with a process called debridement. Debridement may include the removal of dead, damaged, or infected material, such as thick exudate, fibrinous slough, or eschar from a tissue site. In some debridement treatments, a mechanical process is used to remove necrotic tissue. Mechanical processes may include using scalpels or other cutting tools having a sharp edge to cut away the necrotic tissue from the tissue site. Typically, mechanical processes of debriding a tissue site may be painful and may require the application of local anesthetics. Some mechanical processes can create domes or raised nodules, some of which can have the appearance of pimples, in a tissue site. For example, the domes may be raised and have a residual yellow-whitish colored sloughy material on a top surface. These domes can be unsightly, causing potential distress to the patient. The domes can also interfere with the integration of skin grafts following slough and eschar removal.

Debridement may also be performed with an autolytic process. An autolytic process may involve using enzymes and moisture produced by a tissue site to soften and liquefy the necrotic tissue. Typically, a dressing may be placed over a tissue site having necrotic tissue so that fluid produced by the tissue site may remain in place, hydrating the necrotic tissue. Autolytic processes can be pain-free, but autolytic processes are a slow and can take many days. Because autolytic processes are slow, autolytic processes may also involve many dressing changes. Some autolytic processes may be paired with negative-pressure therapy so that, as necrotic tissue hydrates, negative pressure supplied to a tissue site may draw off the removed necrotic tissue. In some cases, a manifold positioned at a tissue site to distribute negative-pressure across the tissue site may become blocked or clogged with necrotic tissue broken down by an autolytic process. If a manifold becomes clogged, negative-pressure may not be able to draw off necrotic tissue, which can slow or stop the autolytic process.

Debridement may also be performed by adding enzymes or other agents to the tissue site. The enzymes can digest tissue. Often, strict control of the placement of the enzymes and the length of time the enzymes are in contact with a tissue site must be maintained. If enzymes are left on the tissue site for longer than needed, the enzymes may remove too much tissue, contaminate the tissue site, or be carried to other areas of a patient. Once carried to other areas of a patient, the enzymes may break down undamaged tissue and cause other complications.

These limitations and others may be addressed by the therapy system 100, which can provide negative-pressure therapy, instillation therapy, and debridement. For example, in some embodiments of the therapy system 100, a negative-pressure source may be fluidly coupled to a tissue site to provide negative pressure to the tissue site for negative-pressure therapy. In some embodiments, a fluid source may be fluidly coupled to a tissue site to provide therapeutic fluid to the tissue site for instillation therapy. In some embodiments, the therapy system 100 may include a debridement tool positioned adjacent to a tissue site. In some embodiments of the therapy system 100, the tissue interface 108 can be a debridement tool. A debridement tool may be used with negative-pressure therapy and instillation therapy to debride areas of a tissue site having necrotic tissue. The debridement tool can improve slough removal, increase wound bed deformation of a tissue site, eliminate unsightly domes, and provide a smoother surface for the integration and taking hold of skin grafts, which can provide a smoother healed tissue surface. In some embodiments, the debridement tool can also be applied in a single layer, reducing the total amount of materials needed to cover the tissue site and protecting the tissue site from contact with a cover.

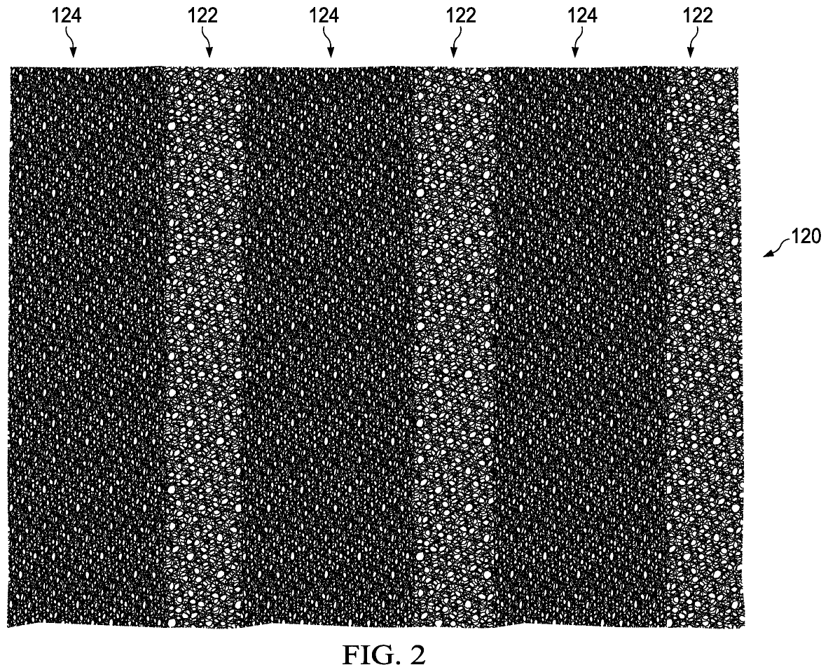

FIG. 2 is a plan view illustrating additional details that may be associated with an example embodiment of a debridement tool 120 that may be used with the therapy system 100 of FIG. 1. The debridement tool 120 or debriding manifold may be an example of the tissue interface 108. In some embodiments, the debridement tool 120 can have a variable density. For example, the debridement tool 120 may include a plurality of first portions 122 and a plurality of second portions 124. In some embodiments, the first portions 122 may have a first density, and the second portions 124 may have a second density. In some embodiments, the second density is greater than the first density. In some embodiments, the debridement tool 120 may be formed from a foam, similar to V.A.C.® GRANUFOAM™ dressing. The first portions 122 can be a V.A.C.® GRANUFOAM™ dressing having a first density, and the second portions 124 can be a V.A.C.® GRANUFOAM™ dressing having a second density. In some embodiments, the second density may be between about 3 times and about 5 times greater than the first density. For example, the first portions 122 may be an uncompressed foam, and the second portions 124 may be a compressed foam having a firmness factor of about 5.

A compressed foam is a foam that is mechanically or chemically compressed to increase the density of the foam at ambient pressure. A compressed foam may be characterized by a firmness factor (FF) that is defined as a ratio of the density of a foam in a compressed state to the density of the same foam in an uncompressed state. For example, a firmness factor (FF) of 5 may refer to a compressed foam having a density that is five times greater than a density of the same foam in an uncompressed state. Mechanically or chemically compressing a foam may reduce a thickness of the foam at ambient pressure when compared to the same foam that has not been compressed. Reducing a thickness of a foam by mechanical or chemical compression may increase a density of the foam, which may increase the firmness factor (FF) of the foam. Increasing the firmness factor (FF) of a foam may increase a stiffness of the foam in a direction that is parallel to a thickness of the foam. For example, increasing a firmness factor (FF) of the debridement tool 120 may increase a stiffness of the debridement tool 120 in a direction that is parallel to a thickness of the debridement tool 120. In some embodiments, a compressed foam may be a compressed V.A.C.® GRANUFOAM™ dressing. V.A.C.® GRANUFOAM™ dressing may have a density of about 0.03 grams per centimeter³ (g/cm³) in its uncompressed state. If the V.A.C.® GRANUFOAM™ dressing is compressed to have a firmness factor (FF) of 5, the V.A.C.® GRANUFOAM™ dressing may be compressed until the density of the V.A.C.® GRANUFOAM™ dressing is about 0.15 g/cm³. V.A.C. VERAFLO™ foam may also be compressed to form a compressed foam having a firmness factor (FF) up to 5. The foam material used to form a compressed foam may be either hydrophobic or hydrophilic. The pore size of a foam material may vary according to needs of the debridement tool 120 and the amount of compression of the first portions 122 and the second portions 124. For example, the first portions 122 formed from an uncompressed foam may have pore sizes in a range of about 400 microns to about 600 microns. If the second portions 124 are formed from a compressed foam, the pore sizes following compression may be smaller than 400 microns.

A compressed foam may also be referred to as a felted foam. As with a compressed foam, a felted foam undergoes a thermoforming process to permanently compress the foam to increase the density of the foam. A felted foam may also be compared to other felted foams or compressed foams by comparing the firmness factor of the felted foam to the firmness factor of other compressed or uncompressed foams. Generally, a compressed or felted foam may have a firmness factor greater than 1.

Generally, if a compressed foam is subjected to negative pressure, the compressed foam exhibits less deformation than a similar uncompressed foam. If the second portions 124 are formed of a compressed foam, the thickness of the second portions 124 may deform less than the first portions 122 that are formed of a comparable uncompressed foam. The decrease in deformation may be caused by the increased stiffness as reflected by the firmness factor (FF). If subjected to the stress of negative pressure, the second portions 124 that are formed of compressed foam may flatten less than the first portions 122 that are formed from uncompressed foam. The degree of compression of the first portions 122 and the second portions 124 can be inversely proportional to the degree of felting. For example, a 10 mm thick piece of foam having a firmness factor of 2 will compress half as much as a 10 mm thick piece of foam having a firmness factor of 1. In some embodiments, the debridement tool 120 may have a thickness of about 8 mm, and if the debridement tool 120 is positioned within a sealed therapeutic space and subjected to negative pressure of about −125 mmHg the debridement tool 120 may compress. The second portions 124 may compress less than the first portions 122. Under negative pressure, the second portions 124 may have a thickness of about 6 mm, and the first portions 122 may have a thickness of about 3 mm. In some embodiments, the first portions 122 may be more compressible than the second portions 124.

In some embodiments, the debridement tool 120 can be formed from a block of foam. An uncompressed block of foam having six sides can be provided. A plurality of channels can be formed in a first surface of the block. For example, the first surface can be cut to form the channels. Cutting can include cutting with a laser-cutting, computer numerical control (“CNC”) hot wire cutting, and pressing the foam block through holes in a plate configured to shear away material and then cleaving the foam. Cutting can also include egg-crating, for example, cutting the foam with a specially designed band saw operable to simultaneously cut the foam at variable depths. In some embodiments, channels may also be formed in a second surface of the block. For example, the second surface can be on an opposite side of the block from the first surface. The channels of the second surface may be aligned with the channels of the first surface. The channels may be parallel, and each channel may run the length or width of the block and have a width or length substantially equal to the width of the first portions 122. In some embodiments, the channels have a square or rectangular shape. Formation of the channels creates a series of parallel walls extending from the first surface of the block of foam. Viewed from a sided perpendicular to the first surface, the first surface may have an undulating topography similar to a square-wave shape. In other embodiments, the channels may be formed having a circular, a triangular, or an amorphous shape, creating a sine-wave, saw-tooth (triangular) wave, or amorphous wave profile, respectively.

Following formation of the channels, the block may be compressed or felted. For example, the first surface and the second surface can be positioned between two plates designed to heat the block. After heating to an optimal temperature for the particular foam, the plates can compress the foam. The plates hold the foam in the compressed state until the foam cools to ambient temperatures, retaining the thickness of the compressed state. In some embodiments, the block may be felted or compressed until the block has a substantially uniform thickness, forming a debridement tool 120 having a substantially uniform thickness. Felting of the block of foam to have a substantially uniform thickness will compress the walls, so that the walls have a greater density than the adjacent channels. After felting, the channels comprise the first portions 122, and the compressed walls comprise the second portions 124. The felting process creates the first portions 122 and the second portions 124 having different densities as more material is compressed into the new volume created by the felting process at the second portions 124 relative to the first portions 122. In other embodiments, the debridement tool 120 may have a slight variation in thickness between the first portions 122 and the second portions 124.

In some embodiments, two or more debridement tools 120 can be assembled for use as a single device. For example, a first debridement tool 120 can be positioned over a second debridement tool 120 so that the first portions 122 and the second portions 124 of the first debridement tool 120 are perpendicular to the first portions 122 and the second portions 124 of the second debridement tool 120. The first debridement tool 120 can be coupled to the second debridement tool 120. For example, the first debridement tool 120 and the second debridement tool 120 can be flame laminated, adhered, hot melted, or further felted together.

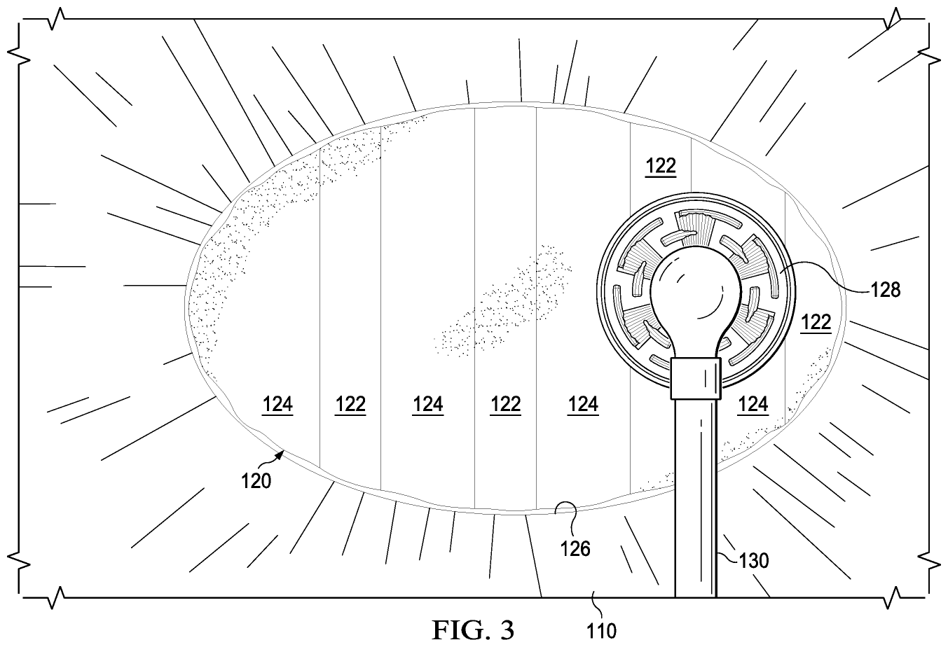

FIG. 3 is a plan view illustrating additional details of the operation of the debridement tool 120 disposed at an exemplary tissue site 126 during a bench testing process. The tissue site 126 may be a test tissue site molded from Dermasol, a highly-elastic thermoplastic elastomer. The debridement tool 120 can be positioned in the tissue site 126 and covered with a cover 110. The cover 110 can be sealed to the tissue surrounding the tissue site 126, periwound tissue, to form a sealed therapeutic environment containing the debridement tool 120. A hole can be formed in the cover 110 over the debridement tool 120 and a dressing interface 128 can be positioned over and sealed around the hole in the cover 110. A tube 130 can couple the dressing interface 128 to the negative-pressure source 102 (not shown). The negative-pressure source 102 can be operated to draw fluid from the tissue site 126 through the debridement tool 120, generating a negative pressure in the sealed therapeutic environment.

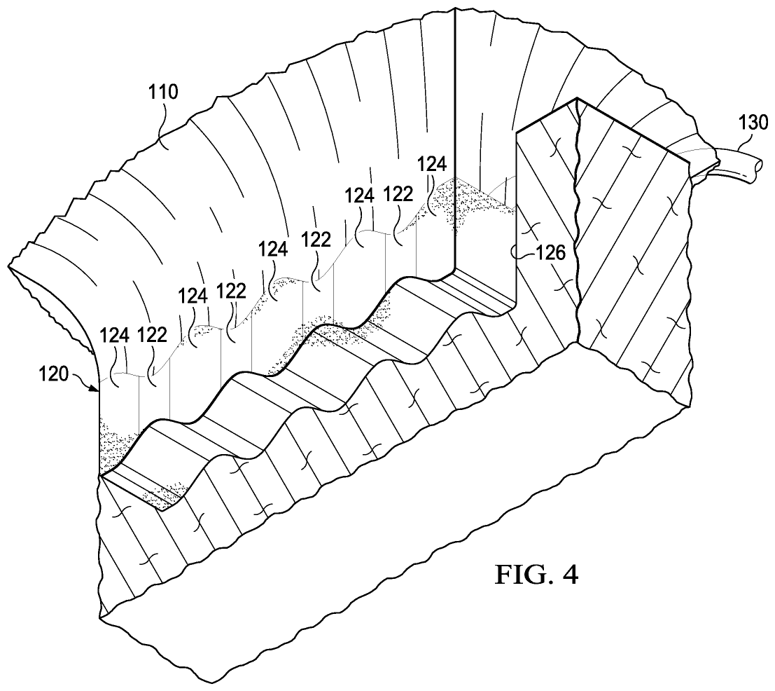

FIG. 4 is a bottom perspective view illustrating additional details of the debridement tool 120 disposed at the exemplary tissue site 126 during negative-pressure therapy of a bench testing process. FIG. 4 may illustrate a moment in time where a pressure in the sealed therapeutic environment may be about 125 mmHg of negative pressure. In some embodiments, the first portions 122 may be an uncompressed foam, and the second portions 124 may be a felted foam having a firmness factor of 5. For example, the second portions 124 can be the equivalent a 50 mm layer of foam compressed to a total thickness of 10 mm. In response to the application of negative pressure, the first portions 122 may compress more than the second portions 124. Under negative pressure, the difference in density between the first portions 122 and the second portions 124 can cause opposite surfaces of the debridement tool 120 to form an alternating waveform shape, for example a sine-wave shape. The surfaces can have a crest near a center of a second portion 124 and a trough near a center of a first portion 122. The surfaces transition between the crest and the trough as the surfaces transition from the more dense second portions 124 to the less dense first portions 122. In some embodiments, the thickness of the first portions 122 during negative-pressure therapy may be less than the thickness of the first portions 122 if the pressure in the sealed therapeutic environment is about the ambient pressure. For example, in some embodiments, the second portions 124 may be 2 mm to 10 mm thicker than the first portions 122.

In some embodiments, negative pressure in the sealed therapeutic environment can generate concentrated stresses in the tissue site 126. For example, the sine-wave pattern developed in the surface of the debridement tool 120 can cause tissue adjacent to the surface of the debridement tool 120 to deform in a similar sine-wave pattern. Areas of tissue adjacent the first portions 122 may deform more than areas of tissue adjacent to the second portions 124, forming concentrated stresses in the tissue transitioning between a crest and a trough of each wave. The concentrated stresses can cause macro-deformation of the tissue site 126 that deforms the tissue site 126.

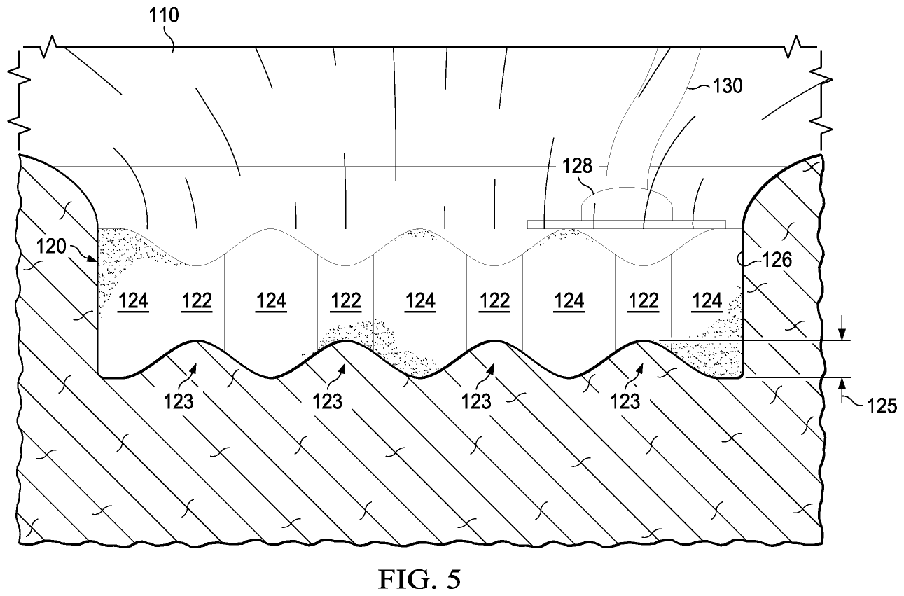

FIG. 5 is a side view of the debridement tool 120 with a portion of the exemplary tissue site 126 shown in section. In the illustrated embodiment, the debridement tool 120 is under approximately 125 mmHg of negative pressure. The surface of the tissue site 126 may have a shape that corresponds to the shape of the deformation of the debridement tool 120. For example, the debridement tool 120 may create deformations 123 in a surface of the tissue site 126. The deformations 123 can have a height 125 from the surface of the tissue site 126. The height 125 of the deformations 123 from the surface of the tissue site 126 can depend, in part, on the difference in densities between the first portions 122 and the second portions 124, a level of the negative pressure in the sealed therapeutic environment, and a relative area of the first portions 122 and the second portions 124.

The height 125 of the deformation 123 over the surrounding tissue may be selected to maximize disruption of the tissue site 126. Generally, the pressure in the sealed therapeutic environment can exert a force that is proportional to the area over which the pressure is applied. At the first portions 122, the force may be concentrated as the resistance to the application of the pressure is less than in the second portions 124. In response to the force generated by the pressure at the first portions 122, the tissue site 126 may be drawn into the first portions 122, creating the deformations 123 until the force applied by the pressure is equalized by the reactive force of the tissue site 126 and the debridement tool 120. In some embodiments where the negative pressure in the sealed therapeutic environment may cause tearing, the relative firmness factors of the first portions 122 and the second portions 124 may be selected to limit the height of the deformations 123 over the surrounding tissue. By controlling the firmness factor of the first portions 122 and the second portions 124, the height 125 of the deformations 123 over the surrounding material of the tissue site 126 can be controlled. In some embodiments, the height 125 of the deformations 123 can vary from zero to several millimeters as the firmness factor of the first portions 122 relative to the firmness factor of the second portions 124 decreases. In an exemplary embodiment, the second portions 124 may have a thickness of about 8 mm. The thickness of the second portions 124 may be about 7 mm under negative pressure. During the application of negative pressure, the thickness of the first portions 122 may be between about 4 mm to about 5 mm, limiting the height 125 of the deformations 123 to about 2 mm to about 3 mm. In another exemplary embodiment, application of negative pressure of between about −50 mmHg and about −350 mmHg, between about −100 mm Hg and about −250 mmHg and, more specifically, about −125 mmHg in the sealed therapeutic environment may reduce the thickness of the second portions 124 having a firmness factor of 3 from about 8 mm to about 3 mm. If the first portions 122 are adjacent to the second portions 124, the height 125 of the deformations 123 may be limited to be no greater than the thickness of the second portions 124 during negative-pressure therapy less the thickness of the first portions 122 under negative-pressure therapy. By controlling the height 125 of the deformations 123 by controlling the firmness factor of the second portions 124, the firmness factor of the first portions 122, or both, the aggressiveness of disruption to the tissue site 126 and tearing can be controlled.

Disruption of the tissue site 126 can be caused, at least in part, by the concentrated forces applied to the tissue site 126 by the differing deformation of the first portions 122 relative to the second portions 124. The forces applied to the tissue site 126 can be a function of the negative pressure supplied to the sealed therapeutic environment and the area of each first portion 122 and each second portion 124. For example, if the negative pressure supplied to the sealed therapeutic environment is about 125 mmHg and the area of each first portion 122 is about 25 mm², the force applied is about 0.07 lbs. If the area of each first portion 122 is increased to about 64 mm², the force applied at each first portion 122 can increase up to 6 times. Generally, the relationship between the area of each first portion 122 and the applied force at each first portion 122 is not linear and can increase exponentially with an increase in area. In some embodiments, the negative pressure applied by the negative-pressure source 102 may be cycled rapidly. For example, negative pressure may be supplied for a few seconds, then vented for a few seconds, causing a pulsation of negative pressure in the sealed therapeutic environment. The pulsation of the negative pressure can pulsate the deformations 123, causing further disruption of the tissue site 126.

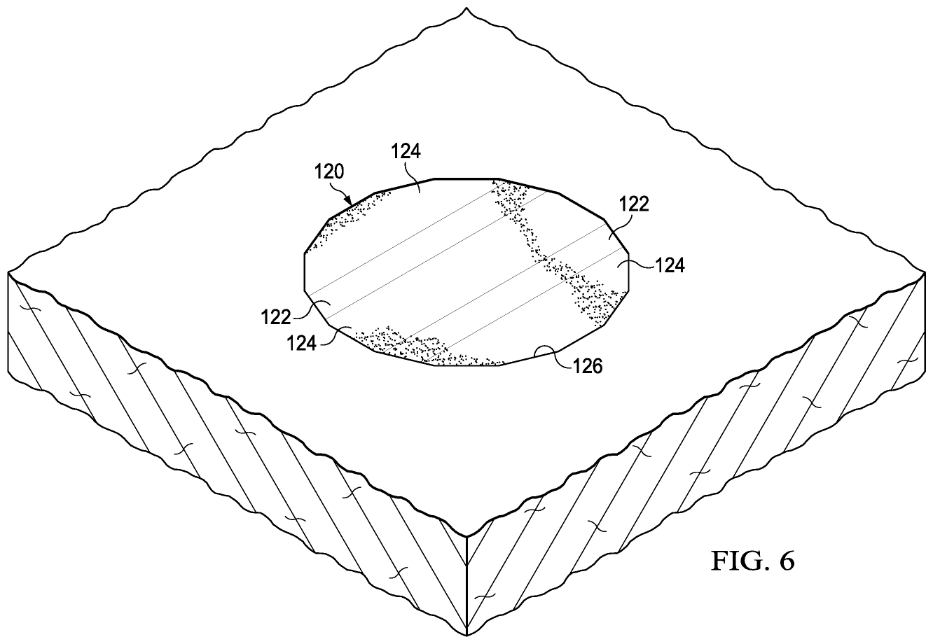

FIG. 6 is a perspective view illustrating additional details of the debridement tool 120 disposed in another exemplary bench testing apparatus. Again, the debridement tool 120 can be disposed at the exemplary tissue site 126. The tissue site 126 may be a test tissue site molded from Dermasol, a highly elastic thermoplastic elastomer. Generally, the tissue site 126 can be substantially filled by the debridement tool 120. In some embodiments, the first portions 122 and the second portions 124 may be vertically oriented relative to a tissue site, and in other embodiments, the first portions 122 and the second portions 124 may be horizontally oriented relative to a tissue site. In still other embodiments, the first portions 122 and the second portions 124 may be oriented at an angle relative to a tissue site.

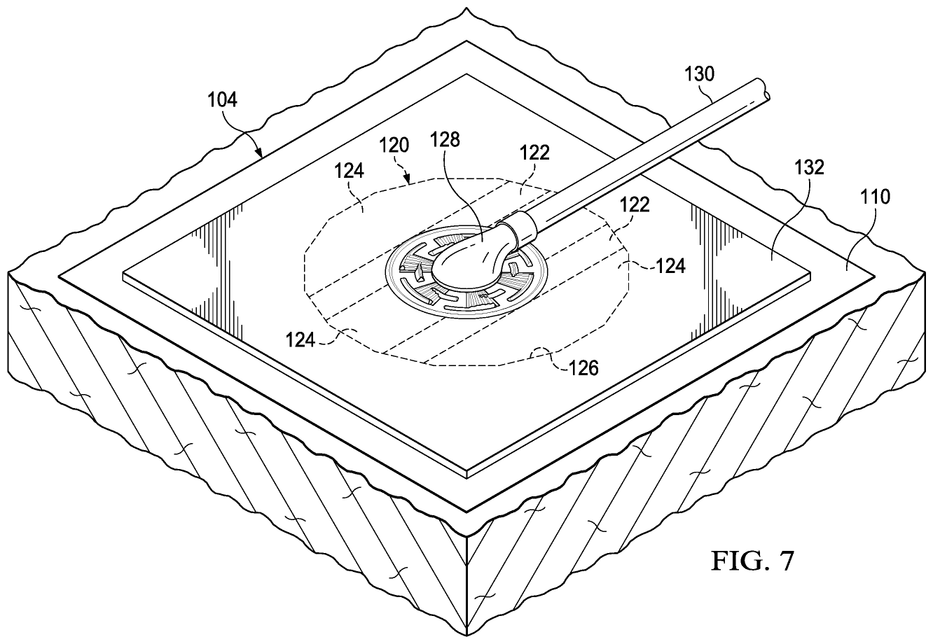

FIG. 7 is a perspective view illustrating additional details that may be associated with some embodiments of the debridement tool 120 of FIG. 6. The debridement tool 120 can be positioned in the tissue site 126 and covered with a support layer, such as a rigid layer 132. The rigid layer 132 can be a layer polymer material having a thickness between about 1 mm and about 5 mm. The rigid layer 132 can be formed from a material having a Young's modulus between about 0.013 gigapascal (“GPa”) and about 0.9 GPa. The size and shape of the rigid layer 132 can be customizable by a user. In some embodiments, the rigid layer 132 may be about 30% larger than the tissue site 126. In some embodiments, the rigid layer 132 may be used as a wound filler. The rigid layer 132 may also be used as a filter layer to limit clogging of the vacuum line by wound material. The debridement tool 120 and the rigid layer 132 can be covered by the cover 110. The cover 110 can be sealed to the tissue surrounding the tissue site 126 to form a sealed therapeutic environment containing the debridement tool 120. A hole can be formed in the cover 110 over the debridement tool 120 and a dressing interface 128 can be positioned over and sealed around the hole in the cover 110. A tube 130 can couple the dressing interface 128 to the negative-pressure source 102 (not shown). The negative-pressure source 102 can be operated to draw fluid from the tissue site 126 through the debridement tool 120.

FIG. 8 is a perspective view of the tissue site 126 illustrating details of tissue deformation produced from the operation of the therapy system 100 with the addition of the rigid layer 132. In the exemplary embodiment, following the application of negative pressure at approximately −125 mmHg through the debridement tool 120, the tissue site 126 was frozen, the cover 110, the rigid layer 132, and the debridement tool 120 were removed, and the resulting deformation of the tissue site 126 was examined. The addition of the rigid layer 132 increased the height 125 of the deformation 123 of the tissue site 126 compared to the debridement tool 120 used without the rigid layer 132.

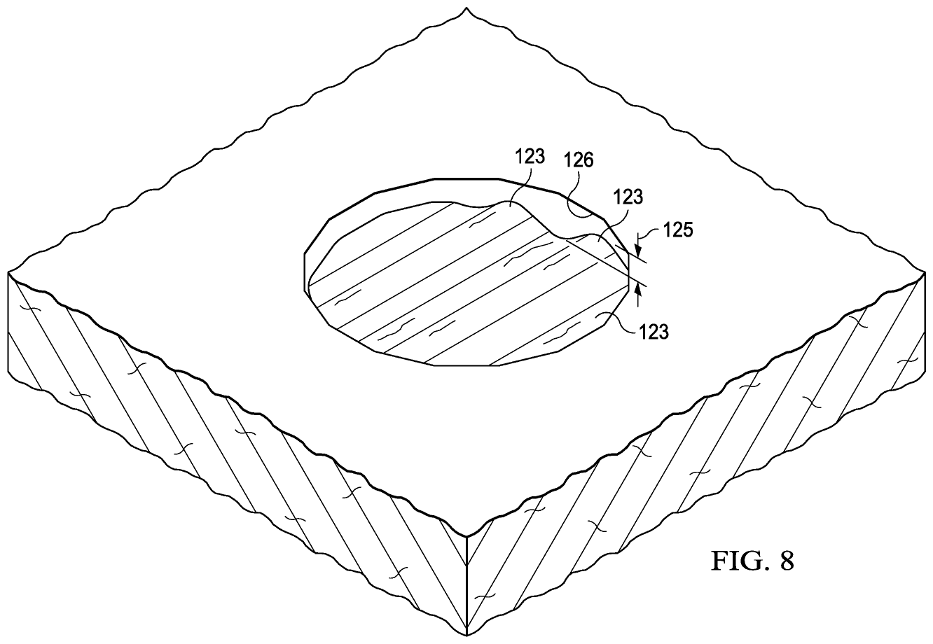

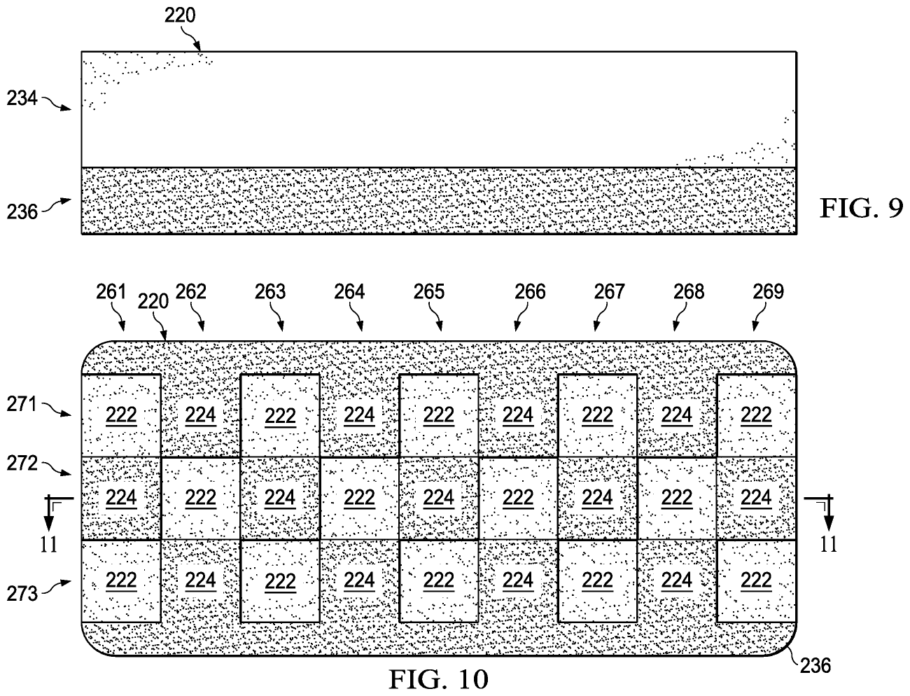

FIG. 9 is a side view illustrating additional details that may be associated with some embodiments of a debridement tool 220 that can be used with some embodiments of the therapy system 100 of FIG. 1. In some embodiments, the debridement tool 220 may be another example of a tissue interface 108. The debridement tool 220 may include a first part 234 and a second part 236. In some embodiments, the first part 234 may be an upper part, and the second part 236 may be a lower part. For example, the debridement tool 220 can be positioned so that the second part 236 is proximate to a tissue site, and the first part 234 is over the second part 236. In some embodiments, the first part 234 may be formed by a foam. For example, the first part 234 can be formed from a compressed foam or a felted foam similar to V.A.C.® GRANUFOAM™ dressing. The first part 234 can have a firmness factor of up to 10. In some embodiments, the second part 236 may have variable densities. For example, the second part 236 may have regions having a first density and regions having a second density. In some embodiments, the first part 234 and the second part 236 can be two layers that are fused together to form a unitary body. For example, the first part 234 can be formed having a first density or firmness factor, and the second part 236 can be formed having variable densities. The first part 234 can be positioned over the second part 236 so that their respective edges are contiguous, and the first part 234 can be coupled to the second part 236. For example, the first part 234 can be fused, adhered, welded, or otherwise joined to the second part 236. In other embodiments, the first part 234 and the second part 236 can be separate layers configured to be positioned separately at a tissue site. For example, the first part 234 can be a manifold or other tissue interface configured to be positioned over the second part 236.

FIG. 10 is a bottom view illustrating additional details that may be associated with some embodiments of the second part 236 of the debridement tool 220. The debridement tool 220 may have a plurality of first portions 222 and a plurality of second portions 224. The plurality of first portions 222 may have a first density, and the plurality of the second portions 224 may have a second density. In some embodiments, the first density may be greater than the second density. In other embodiments, the first density may be less than the second density. The second portions 224 may be a felted foam or a compressed foam with a firmness factor of about 1.5 to about 10, for example, 1.5, 2, 3, 5, 7.5, or 10. The first portions 222 may be a non-felted foam, uncompressed foam, or a foam having a firmness factor less than the firmness factor of the second portions 224. For example, the second part 236 may be formed by the first portions 222 with the first density and the second portions 224 with the second density. In some embodiments, the first part 234 can have a density similar to the density of the second portions 224.

The plurality of first portions 222 and the plurality of second portions 224 may be arrayed across the surface of the debridement tool 220 to form a cross-hatched or grid pattern. For example, a surface of the debridement tool 220 can be arrayed in a series of repeating columns and rows. In the illustrated embodiment, the surface of the debridement tool 220 is arranged with nine columns: a first column 261, a second column 262, a third column 263, a fourth column 264, a fifth column 265, a sixth column 266, a seventh column 267, an eight column 268, and a ninth column 269; and three rows: a first row 271, a second row 272, and a third row 273. The columns and rows can be perpendicular to each other and intersecting. In some embodiments, each first portion 222 can be positioned so that a second portion 224 is disposed between adjacent first portions 222. Similarly, each second portion 224 can be positioned so that a first portion 222 is disposed between adjacent second portions 224. As a result, a first portion 222 can disposed in the position where the first column 261 intersects the first row 271, and a second portion 224 can be disposed in the position where the second column 262 intersects the first row 271 and in the position where the first column 261 intersects the second row 272.

Each first portion 222 may have a pitch in a first direction parallel to a length and in a second direction parallel to a width between adjacent centers of the first portions 222. In some embodiments, the pitch of the first portions 222 may be equal to twice a length of a first portion 222. Similarly, each second portion 224 may have a pitch between adjacent centers of the second portions 224 equal to twice a length of a second portion 224. In other embodiments, the pitch of the repeating portions can be greater or lesser and oriented at an angle to both the length and width of the debridement tool 220.

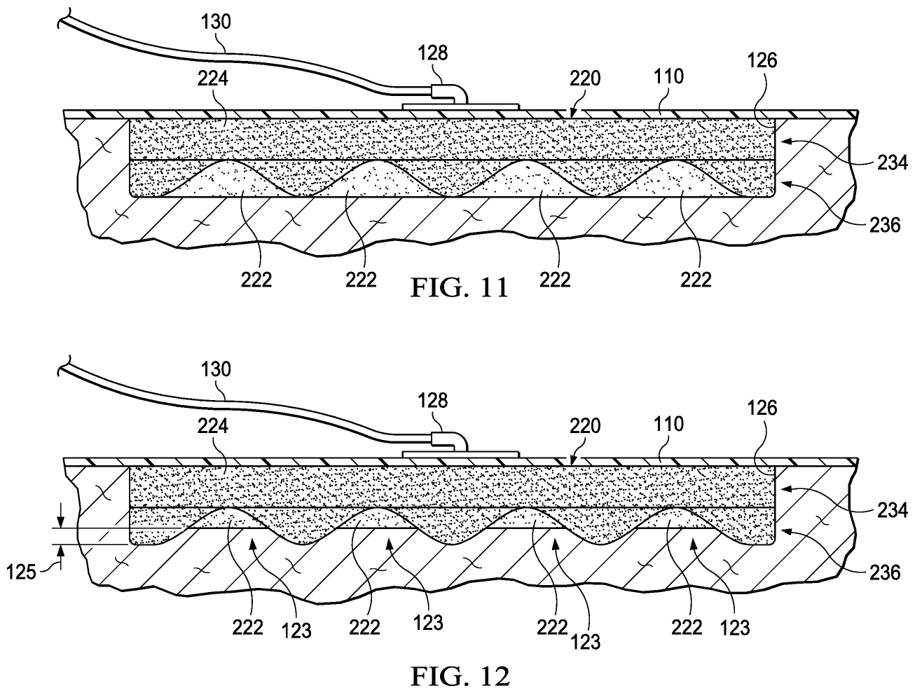

FIG. 11 is a sectional view of the debridement tool 220 disposed at the tissue site 126 illustrating additional details that may be associated with some embodiments. The debridement tool 220 can be positioned adjacent to or proximate to the tissue site 126. The debridement tool 220 can be positioned so that the second part 236 having the first portions 222 and the second portions 224 are disposed adjacent to a surface of the tissue site 126. The debridement tool 220 can be covered with a cover 110. The cover 110 can be sealed to the tissue surrounding the tissue site 126 to form a sealed therapeutic environment containing the debridement tool 220. In some embodiments, the first part 234 may extend vertically past an edge of the tissue site 126, holding the cover 110 spaced from the edge of the tissue site 126. A hole can be formed in the cover 110 over the debridement tool 120 and a dressing interface 128 can be positioned over and sealed around the hole in the cover 110. A tube 130 can couple the dressing interface 128 to the negative-pressure source 102 (not shown). The negative-pressure source 102 can be operated to draw fluid from the tissue site 126 through the debridement tool 120.

FIG. 12 is a sectional view of the debridement tool 220 disposed at the tissue site 126 after the application of negative pressure. In operation, a negative pressure may be supplied to the sealed therapeutic environment, and the debridement tool 220 may contract from a relaxed position illustrated in FIG. 11 to a contracted position illustrated in FIG. 12. As illustrated in FIG. 12, the first portions 222 contract more than the second portions 224 drawing tissue away from a surface of the tissue site 126 to form deformations 123. In the illustrated embodiment, the deformations 123 can be drawn away from the surface of the tissue site 126 to the height 125. In some embodiments, the height 125 can be limited, in part, by the first part 234. The first part 234 can have a density similar to the density of the second portions 224.

In some embodiments, the debridement tool 220 can be formed from a block of foam. An uncompressed block of foam having six sides can be provided. The uncompressed block of foam can be felted using a felting tool configured to provide non-uniform compression to the block of foam. The felting tool can introduce areas of greater relative density in a first operation. For example, the felting tool may form parallel strips of the first portions 222 having a first density that are adjacent to parallel strips of the second portions 224 having a second density that is greater than the first density. The block of foam can be rotated ninety degrees relative to the felting tool and a second non-uniform compression can be performed on the block of foam. For example, the felting tool may form parallel strips of the first portions 222 having the first density that are adjacent to parallel strips of the second portions 224 having the second density that is greater than the first density. The parallel strips of the second felting process may be perpendicular to the parallel strips of the first felting process, resulting in a grid pattern of the first portions 222 and the second portions 224 as shown in FIG. 10. The block of foam can then be planed to create a uniform thickness of the debridement tool 220. In alternative embodiments, multiple felting tools operable to provide various patterns of non-uniform compression may be used.

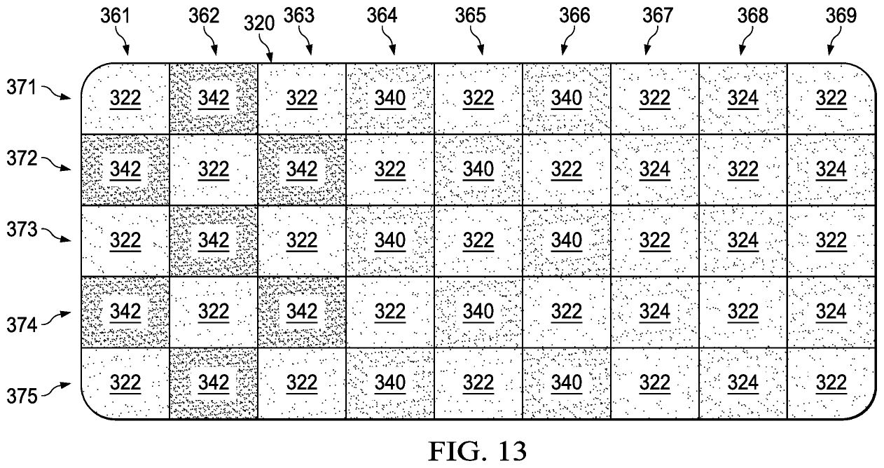

FIG. 13 is a bottom view illustrating additional details that may be associated with some embodiments of a debridement tool 320 that may be used with the therapy system 100 of FIG. 1. The debridement tool 320 or debriding manifold can be another example of the tissue interface 108. In some embodiments, the debridement tool 320 can have more than two different densities. For example, the debridement tool 320 may include a plurality of first portions 322, a plurality of second portions 324, a plurality of third portions 340, and a plurality of fourth portions 342. The first portions 322, the second portions 324, the third portions 340, and the fourth portions 342 may each have a different density or firmness factor. In some embodiments, the first density may be less than the second density, the second density may be less than the third density, and the third density may be less than the fourth density. In some embodiments, the first portions 322 may be un-felted foam having a firmness factor of 1. The second portions 324 may be a felted foam having a firmness factor of 2. The third portions 340 may be a felted foam having a firmness factor of 5, and the fourth portions 342 may be a felted foam having a firmness factor of 10. In other embodiments, the first portions 322, the second portions 324, the third portions 340, and the fourth portions 342 may have different firmness factors. In some embodiments, the firmness factors used to form the first portions 322, the second portions 324, the third portions 340, and the fourth portions 342 can range from about 1 to about 10.

In some embodiments, the first portion 322, the second portions 324, the third portions 340, and the fourth portions 342 may be arrayed across the surface of the debridement tool 320 to form a cross-hatched or grid pattern. For example, the surface of the debridement tool 320 can be arrayed in a series of repeating columns and rows. In the illustrated embodiment, the surface of the debridement tool 320 is arranged with nine columns: a first column 361, a second column 362, a third column 363, a fourth column 364, a fifth column 365, a sixth column 366, a seventh column 367, an eighth column 368, and a ninth column 369; and five rows: a first row 371, a second row 372, a third row 373, a fourth row 374, and a fifth row 375. The columns and rows can be perpendicular to each other and intersecting. In some embodiments, each first portion 322 can be positioned so that one of a second portion 324, a third portion 340, or a fourth portion 342 is disposed between adjacent first portions 322. Similarly, each second portion 324, each third portion 340, and each fourth portion 342 can be positioned so that a first portion 322 is disposed between adjacent second portions 324, adjacent third portions 340, and adjacent fourth portions 342. As a result, a first portion 322 can disposed in the position where the first column 361 intersects the first row 371, and a fourth portion 342 can be disposed in the position where the second column 362 intersects the first row 371 and in the position where the first column 361 intersects the second row 372. In some embodiments, a first portion 322 can disposed in the position where the fifth column 365 intersects the first row 371, and a third portion 340 can be disposed in the position where the sixth column 366 intersects the first row 371 and in the position where the fifth column 365 intersects the second row 372. A first portion 322 can disposed in the position where the seventh column 367 intersects the first row 371, and a second portion 324 can be disposed in the position where the eighth column 368 intersects the first row 371 and in the position where the seventh column 367 intersects the second row 372.

Each first portion 322 may have a pitch in a first direction parallel to a length and in a second direction parallel to a width between adjacent centers of the first portions 322. In some embodiments, the pitch of the first portions 322 may be equal to twice a length of a first portion 322. Similarly, each second portion 324 may have a pitch between adjacent centers of the second portions 324 equal to twice a length of a second portion 324; each third portion 340 may have a pitch between adjacent centers of the third portions 340 equal to twice a length of a third portion 340; and each fourth portion 342 may have a pitch between adjacent centers of the fourth portions 342 equal to twice a length of a fourth portion 342. In other embodiments, the pitch of the repeating portions can be greater or lesser and oriented at an angle to both the length and width of the debridement tool 320. In some embodiments, each portion of the first portions 322, the second portions 324, the third portions 340, and the fourth portions 342 can be a square having a length between about 5 mm and about 10 mm. In other embodiments, each portion of the first portions 322, the second portions 324, the third portions 340, and the fourth portions 342 can be triangular, circular, rectangular, ovoid, or amorphous and can have a major dimension between about 5 mm and about 10 mm.

In some embodiments, the first portions 322, the second portions 324, the third portions 340, and the fourth portions 342 can create discrete gradients of tissue deformation at the tissue site. For example, the difference in densities between the fourth portions 342 and the first portions 322 adjacent to the fourth portions 342 is greater than the difference in densities between the third portions 340 and the first portions 322 and the second portions 324 and the first portions 322. The fourth portions 342 will compress less under negative pressure than the first portions, 322, the second portions 324, and the third portions 340. The first portions 322 will compress more under negative pressure than the second portions 324, the third portions 340, and the fourth portions 342. If negative pressure is applied, the debridement tool 320 can have the largest difference in thickness between the first portions 322 and the fourth portions 342, with the second portions 324 having the smallest difference in thickness from the first portions 322. The difference in thickness under negative pressure can cause the adjacent tissue to deform in a similar manner. The tissue adjacent the area of the debridement tool 320 having the first portions 322 and the fourth portions 342 can exhibit greater deformation than the tissue adjacent the area of the debridement tool 320 having the first portions 322 and the second portions 324. By selecting the firmness factor and the location of the first portions 322, the second portions 324, the third portions 340, and the fourth portions 342 discrete areas of a tissue site can be deformed to greater or lesser heights. In some embodiments, the shapes of the first portions 322, the second portions 324, the third portions 340, and the fourth portions 342 can be selected to address a particular tissue site. For example, the first portions 322, the second portions 324, the third portions 340, and the fourth portions 342 can be arrayed in a radial pattern, a linear pattern, a checkerboard pattern, a diagonal pattern

In other embodiments, the first portions 322, the second portions 324, the third portions 340, and the fourth portions 342 can be disposed in other patterns. For example, the second portions 324, the third portions 340, and the fourth portions 342 can be adjacent to each other without intervening first portions 322. In other embodiments, the portions can be arrayed to create areas of increasing or decreasing density. For example, a fourth portion 342 having a firmness factor of 10 can be surrounded by the third portions 340 having a firmness factor of 5. The third portions 340 can be surrounded by the second portions 324 having a firmness factor of 2, and the second portions 324 can be surrounded by the first portions 322 having a firmness factor of 1. In other embodiments, the portions can be arrayed as needed to address particular types of tissue sites and therapies.

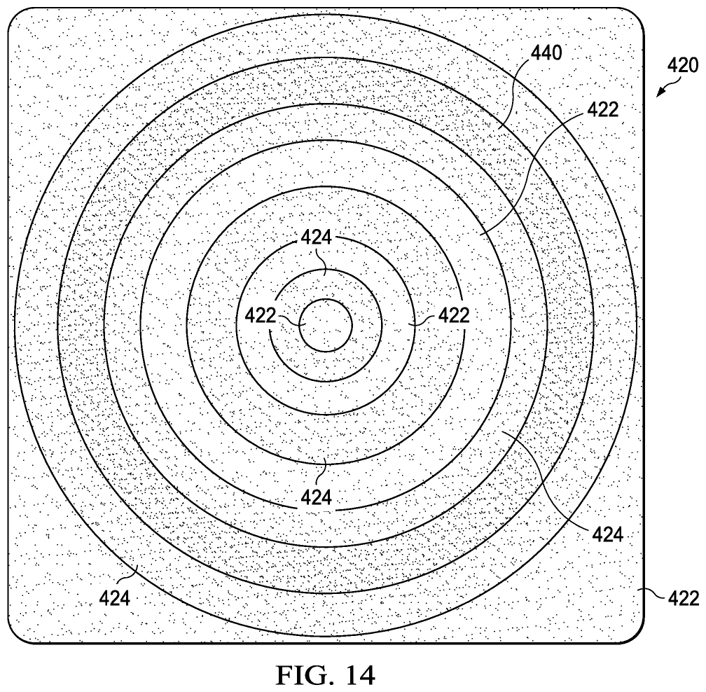

FIG. 14 is a bottom view illustrating additional details that may be associated with some embodiments of a debridement tool 420 that may be used with the therapy system 100 of FIG. 1. The debridement tool 420 or debriding manifold can be another example of the tissue interface 108. The debridement tool 420 may have a plurality of first portions 422, a plurality of second portions 424, and a plurality of third portions 440. The first portions 422, the second portions 424, and the third portions 440 may be arranged in a concentric pattern. For example, the first portions 422 may be disposed at a center of the debridement tool 420. In some embodiments, the first portion 422 located at the center of the debridement tool 420 may be in the shape of a circle. The first portion 422 may be surrounded by a second portion 424, forming a ring around the first portion 422. In some embodiments, a ring of the first portion 422 may surround the ring of the second portion 424, which may similarly be surrounded by a ring of the second portion 424. The alternating rings of the first portion 422 and the second portion 424 may form a target-like pattern in the debridement tool 420. In some embodiments, a ring of the third portion 440 can disposed in the debridement tool 420. For example, a ring of the third portion 440 can be disposed between two rings of the second portion 424. In other embodiments, the first portion 422 may not be in the center, and the order of the rings may be different (not shown). In some embodiments, the first portions 422, the second portions 424, and the third portions 440 can have different densities. For example, in some embodiments, the first portions 422 can have a firmness factor of 1, the second portions 424 can have a firmness factor of 5, and the third portions 440 can have a firmness factor of 10. In other embodiments, each of the first portions 422, the second portions 424, and the third portions 440 can have a different firmness factor.

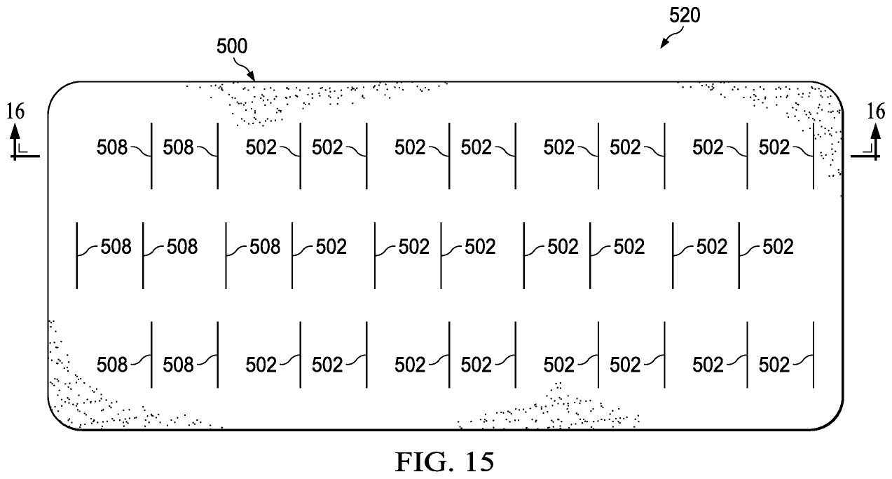

FIG. 15 is a bottom view illustrating additional details that may be associated with some embodiments of the manufacturing process of the debridement tool 520. In some embodiments, a foam block 500 having a firmness factor of 1, an un-felted foam, may be provided. A plurality of first cuts 502 can be made into a surface of the block 500. The first cuts 502 can be made with a laser-cutting, CNC hot wire cutting, and pressing the foam block through holes in a plate configured to shear away material. Cutting can also include egg-crating, for example, cutting the foam with a specially designed band saw operable to simultaneously cut the foam at variable depths. Each of the first cuts 502 may be oriented parallel to an edge of the block 500 and to each other. For example, each of the first cuts 502 may be oriented parallel to a width of the block 500. In other embodiments, the first cuts 502 may not be parallel to a width of the block 500 or to each other.

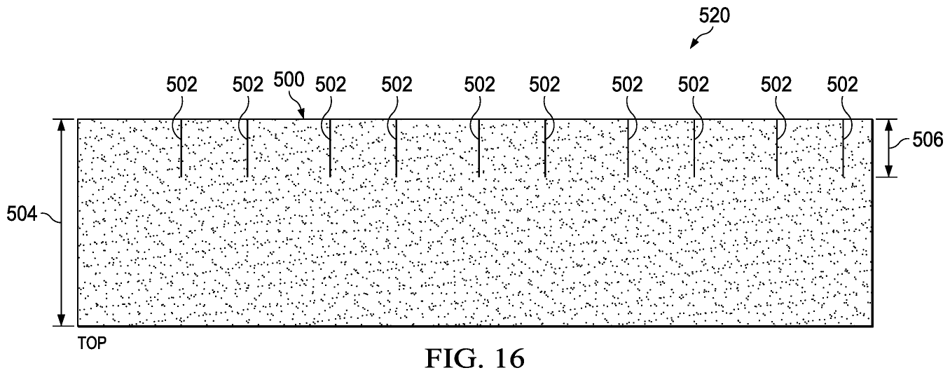

FIG. 16 is a section view of the block 500 of FIG. 15 taken along line 16-16, illustrating additional details that may be associated with some example embodiments of the manufacturing process of the debridement tool 520. The block 500 may have a thickness 504. In some embodiments, each of the plurality of first cuts 502 may have a depth 506 that is less than the thickness 504 of the block 500.

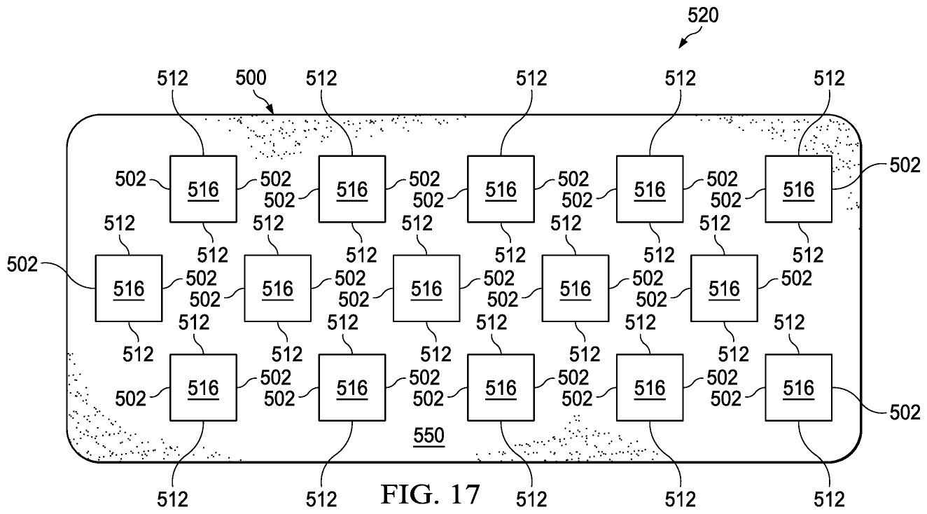

FIG. 17 is a bottom view illustrating additional details that may be associated with some embodiments of the manufacturing process of the debridement tool 520. In some embodiments, the block 500 may have a plurality of second cuts 512. The second cuts 512 can be made with a laser-cutting, CNC hot wire cutting, and pressing the foam block through holes in a plate configured to shear away material. Cutting can also include egg-crating, for example, cutting the foam with a specially designed band saw operable to simultaneously cut the foam at variable depths. In some embodiments, the plurality of second cuts 512 may be parallel to an edge of the block 500 and to each other. For example, the plurality of second cuts 512 can be parallel to a length of the block 500 and to each other. An end of each second cut 512 may intersect an adjacent end of a first cut 502. For example, the block 500 can be rotated ninety degrees following formation of the plurality of first cuts 502 to make the plurality of second cuts 512. The depths of the second cuts 512 may be substantially equal to the depth 506 of the first cuts 502. In some embodiments, the first cuts 502 and the second cuts 512 form protrusions or islands 516. Each of the islands 516 may be separated from adjacent material 550 of the block 500 along a length and width of each island 516. In some embodiments, the first cuts 502 and the second cuts 512 can be performed in a single operation or simultaneously.

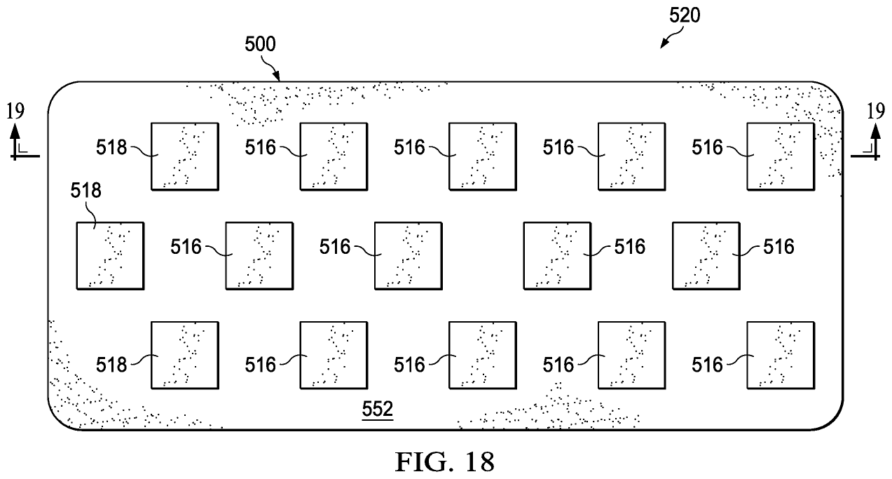

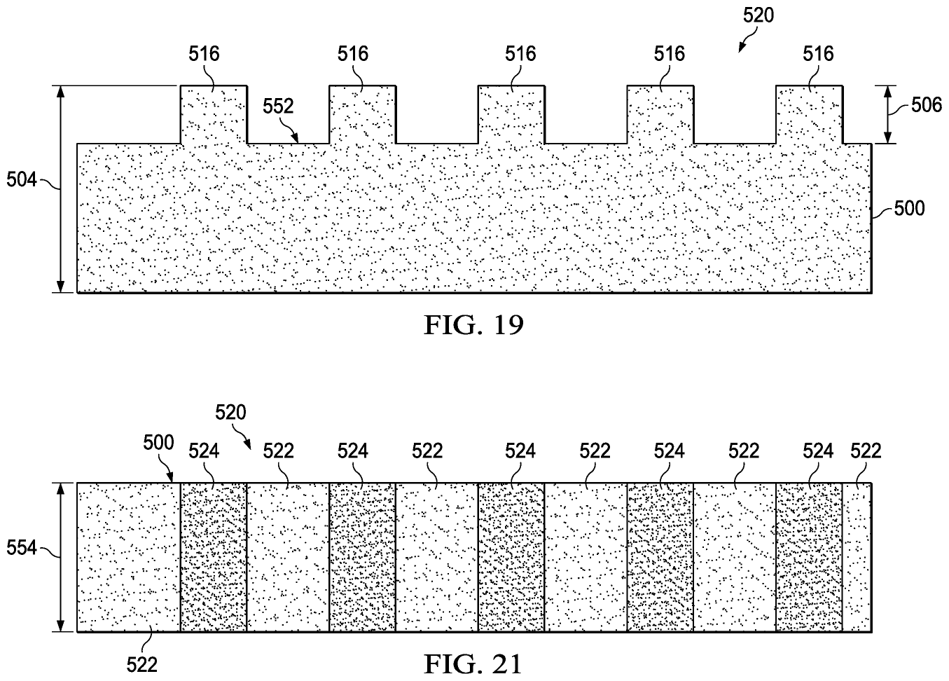

FIG. 18 is a bottom view illustrating additional details that may be associated with some embodiments of the manufacturing process of the debridement tool 520. In some embodiments, the adjacent material 550 can be cleaved, laser-cut, CNC hot wire cut, and pressed through holes in a plate configured to shear away material from the foam block 500. Cutting can also include egg-crating the foam block 500. For example, the block 500 can be cut to remove the adjacent material 550, leaving only the islands 516 and a surface 552 of the block 500. The surface 552 of the block 500 may be separated from a parallel surface of the islands 516. In other embodiments, the islands 516 can be cleaved from the block 500, leaving holes in the block 500. FIG. 19 is a section view of the block 500 of FIG. 18 taken along line 19-19, illustrating additional details that may be associated with some example embodiments of the manufacturing process of the debridement tool 520. As illustrated, the surface 552 can be separated from the parallel surface of the islands 516 by the depth 506, leaving the islands 516. In some embodiments, the islands 516 have a square or rectangular shape. Formation of the islands 516 creates a series of parallel walls extending from the surface 552 of the block 500. Viewed from a sided perpendicular to the surface 552, the block 500 may have an undulating topography similar to a square-wave shape. In other embodiments, the islands 516 may be formed having a circular, a triangular, or an amorphous shape, and can create a sine-wave, saw-tooth (triangular) wave, or amorphous wave profile.

FIG. 20 is a bottom view illustrating additional details that may be associated with some embodiments of the manufacturing process of the debridement tool 520. After removal of the adjacent material 550, the block 500 may be felted to form a debridement tool 520 having first portions 522 and second portions 524. The islands 516 may be compressed into the surface 552, forming the second portions 524 having a density greater than the surrounding portions of the first portions 522. Preferably, the block 500 may be compressed to a uniform thickness so that the surface 552 and the parallel surface of the islands 516 substantially occupies the same plane, forming a surface of the debridement tool 520 that is substantially uniform. The debridement tool 520 may be similar to the debridement tool 220, having opposite areas of greater and lesser density.

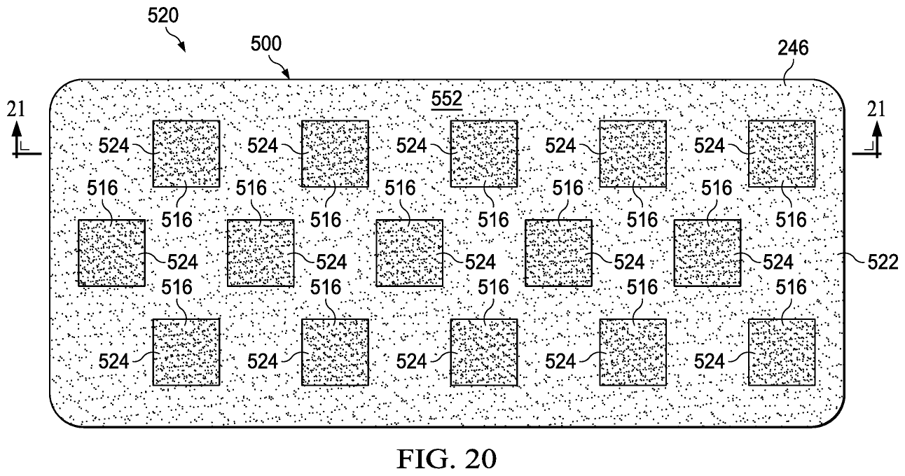

FIG. 21 is a section view of the block 500 taken along line 21-21 of FIG. 20 illustrating additional details that may be associated with some example embodiments of the manufacturing process of the debridement tool 520. In some embodiments, the block 500 is compressed so that the debridement tool 520 has a thickness 554 that is less than the thickness 504 of the block 500. As shown in FIG. 21, the islands 516 can form the more dense areas of the second portions 524. The areas of the adjacent material 550 that are removed adjacent to the islands 516 can form the less dense areas of the first portions 522. The depth 506 can determine the relative densities of the first portions 522 and the second portions 524. The greater the depth 506, the greater the difference in densities of the first portion 522 and the second portions 524. The depth 506 determines how much adjacent material 550 is removed from the block 500 and how much material remains in the islands 516 that must be compressed into the thickness of the debridement tool 520.

In some embodiments, after removal of the adjacent material 550, some of the islands 516 can be cleaved. Cleaving of the islands 516 can create two or more different types of islands 516 differentiated by height from the surface 552. For example, some islands 516 can have a height equal to the depth 506, and some islands 516 can have a second height from the surface 552 that is less than the depth 506. After felting, the islands 516 having a height equal to the depth 506 will be denser than the islands 516 having the second height. Both sets of islands 516 will have a greater density than the surrounding portions of the block 500 removed to the surface 552.

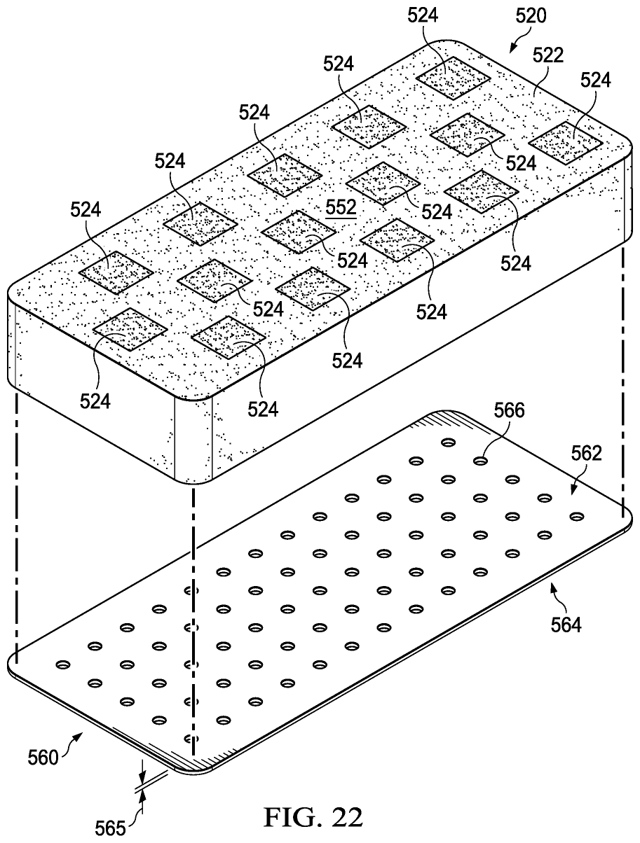

FIG. 22 is a perspective assembly view illustrating additional details of a buffer layer 560 that can be used with some embodiments of the debridement tools described herein. For example, the buffer layer 560 can be used with the debridement tool 520 in the therapy system 100 of FIG. 1. In some embodiments, a buffer layer 560 can have a first side 562 and a second side 564. The first side 562 may be disposed adjacent to the debridement tool 520 and the second side 564 may be configured to be positioned adjacent to a tissue site. The buffer layer 560 may be a film formed from polyurethane or polyethylene. In some embodiments, the buffer layer 560 may have a thickness 565 of about 2 mm to about 10 mm. The buffer layer 560 can include a plurality of perforations 566. In some embodiments, each perforation 566 of the plurality of perforations 566 may be equidistantly spaced from adjacent perforations 566. In other embodiments, each perforation 566 of the plurality of perforations 566 may not be equidistantly spaced from adjacent perforations 566. The perforations 566 may have a pitch from adjacent perforations of about 2 mm to about 20 mm. In some embodiments, the perforations 566 may be circular. In other embodiments, each perforation 566 may have a square, rectangular, triangular, ovular, or amorphous shape. Each perforation 566 may have an average effective diameter of about 5 mm to about 10 mm. An effective diameter may be a diameter of a circular area having the same surface area as non-circular area. In some embodiments, the buffer layer 560 may be coupled to the tissue-facing side of the debridement tool 520. For example, the buffer layer 560 can be laminated, flame-laminated, hot melted, adhered, welded, or otherwise secured to a surface of the debridement tool 520. The buffer layer 560 can further enhance the resistance of the debridement tool 520 to dome formation and tissue ingrowth. The buffer layer 560 may be further combined with coatings or additives, such as citric acid or silver nitrate. The addition of citric acid or silver nitrate can increase the ability of the buffer layer 560 to resist biofilm colony formation. In other embodiments, the buffer layer 560 can include a coating or an additive for drug delivery. For example, the buffer layer 560 may be used to deliver a locally acting pain-relieving agent, such as lidocaine or ketoprophen, etc.

Each of the debridement tool 120, 220, 320, 420, and 520 can have a ratio of felted to non-felted foam of about 1:1 up to 1:10. Each of the debridement tool 120, 220, 320, 420, and 520 can have an average pore size ranging from about 100 microns to about 600 microns. In some embodiments, the felted portions of each of the debridement tool 120, 220, 320, 420, and 520 can have about 20 pores per inch. In some embodiments, each of the debridement tool 120, 220, 320, 420, and 520 can have a minimum firmness factor of about 1 and a maximum firmness factor of about 10. In some embodiments, the maximum firmness factor may be about 1.5 or 5.

The systems, apparatuses, and methods described herein may provide significant advantages. For example, a debridement tool as described herein may provide an improved method for wound bed formation, wound surface elongation, and slough removal without the unsightly formation of domes. The debridement tool may provide a smoother surface for skin grafts to integrate to and take hold and the resulting tissue may have smoother appearance at the conclusion of healing. The debridement tool described herein can also create discrete areas of deformation in the tissue site. Some embodiments of the debridement tool may also increase the surface area of the tissue site in contact with the debridement tool and increase the resulting elongation in the wound bed and at the wound margins. In some embodiments, the debridement tool may also eliminate the need for multiple layers, allowing users to apply one layer to the tissue site while protecting the wound bed from contact with an adhesive drape.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations the dressing 104, the container 106, or both may be eliminated or separated from other components for manufacture or sale. In other example configurations, the controller 112 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A dressing for treating a tissue site with negative pressure, the dressing comprising:
a debriding manifold comprising a plurality of first regions having a first density, and a plurality of second regions having a second density less than the first density; and
a cover configured to be disposed over the debriding manifold and comprising a perimeter extending beyond the debriding manifold.

2. The dressing of claim 1, wherein the second regions are recessed relative to the first regions.

3. The dressing of claim 1, wherein the first regions comprise a first material and the second regions comprise a second material.

4. The dressing of claim 1, wherein the plurality of first regions and the plurality of second regions are alternately distributed in an array through the debriding manifold.

5. The dressing of claim 1, wherein the debriding manifold comprises foam.

6. The dressing of claim 1, wherein the debriding manifold comprises open-cell foam.

7. The dressing of claim 1, wherein the debriding manifold comprises reticulated foam.

8. The dressing of claim 1, further comprising a support layer configured to be disposed between the debriding manifold and the cover, the support layer having a Young's modulus between about 0.013 gigapascal (“GPa”) and about 0.9 GPa.

9. The dressing of claim 1, further comprising a buffer layer having a first side and a second side, the first side disposed adjacent to the debriding manifold and the second side configured to face the tissue site.

10. The dressing of claim 9, wherein the buffer layer is laminated to the second side of the debriding manifold.

11. The dressing of claim 9, wherein the buffer layer is configured to resist ingrowth from the tissue site.

12. The dressing of claim 9, wherein the buffer layer is perforated.

13. The dressing of claim 9, wherein the buffer layer comprises a film of polyurethane.

14. The dressing of claim 9, wherein the buffer layer comprises a film of polyethylene.

15. The dressing of claim 9, wherein the buffer layer is at least partially infused with citric acid.

16. The dressing of claim 9, wherein the buffer layer is at least partially infused with silver nitrate.

17. The dressing of claim 9, wherein the buffer layer is at least partially infused with a pain-relieving agent.

18. The dressing of claim 17, wherein the pain-relieving agent is lidocaine.

19. The dressing of claim 17, wherein the pain-relieving agent is ketoprophen.

20. A method of manufacturing a dressing for negative-pressure treatment, the method comprising:
providing a manifold having a first side and a second side;
cutting a first wave pattern into the second side of the manifold;
rotating the manifold ninety degrees and cutting a second wave pattern into the second side of the manifold; and
simultaneously compressing and heating at least the second side of the manifold.

21. The method of claim 20, wherein the manifold comprises foam.

22. The method of claim 20, wherein the first wave pattern and the second wave pattern are a square wave pattern.

23. The method of claim 20, wherein the first wave pattern and the second wave pattern are a triangular wave pattern.

24. The method of claim 20, wherein the first wave pattern and the second wave pattern are a sine wave pattern.

25. A dressing for treating a tissue site with negative pressure, the dressing comprising:
a debridement manifold having a first section and a second section, the second section positioned adjacent to the first section;
wherein the second section of the debridement manifold comprises a plurality of first regions and a plurality of second regions, the plurality of first regions having a greater density than the plurality of second regions.

26. The dressing of claim 25, wherein the first section of the debridement manifold is a first layer and the second section of the debridement manifold is a second layer.

27. The dressing of claim 26, wherein the first layer has a first side and a second side, the second side configured to face the second layer.

28. The dressing of claim 27, wherein the second side of the first layer comprises a first plurality of protrusions.

29. The dressing of claim 27, wherein the second layer has a first side and a second side, the first side configured to face the second side of the first layer.

30. The dressing of claim 25, wherein the plurality of first regions and the plurality of second regions are square-shaped.

31. The dressing of claim 25, wherein the plurality of first regions and the plurality of second regions are triangle-shaped.

32. The dressing of claim 25, wherein the plurality of first regions and the plurality of second regions are wave-shaped.

33. (canceled)
